(19) United States
(12) Patent Application Publication
(10) Pub. No.: US 2002/0197225 A1
(43) Pub. Date: Dec. 26, 2002

(54) Hair composition comprising a polysaccharide grafted with a polysiloxane, and a fixing polymer

(76) Inventors: Franck Giroud, Clichy (FR); Christine Dupuis, Paris (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/094,809

(22) Filed: Mar. 12, 2002

(30) Foreign Application Priority Data
Mar. 13, 2001 (FR) 01 03404

(51) Int. Cl. A61K 7/06; A61K 7/11

(52) U.S. Cl. 424/70.12

(57) ABSTRACT

A cosmetic hair composition comprising in a cosmetically acceptable medium, (a) at least one polymer comprising a polysaccharide skeleton grafted with at least one group comprising at least one polysiloxane; and (b) at least one non-silicone, non-cellulosic fixing polymer. The invention also relates to a method of making the composition, a cosmetic hair process comprising the application of this composition to hair, and also to methods for fixing and/or holding a hairstyle.

The invention relates to a cosmetic hair composition comprising: (a) at least one polymer comprising a polysaccharide skeleton grafted with at least one group comprising at least one polysiloxane, and (b) at least one non-silicone, non-cellulosic fixing polymer. The invention also relates to a cosmetic hair process comprising the application of this composition to the hair, and also to its use for fixing and/or holding the hairstyle.

Among the most common hair products on the cosmetic market, especially those intended for shaping and/or holding the hairstyle, are spray compositions consisting essentially of a solution, which is usually alcoholic or aqueous, and of one or more materials.

When the compositions are intended for fixing and/or holding the hairstyle, these materials are generally polymer resins, whose function is to form welds between hairs, also known as fixing materials, mixed with various cosmetic adjuvants. These compositions are generally packaged either in a suitable aerosol container pressurized by using a propellant, or in a pump-dispenser bottle.

Hair mousses or gels, which are generally applied to wet hair before blow-drying or shaping the hairstyle, are also known. These gels or mousses can also contain polymer resins.

However, these hair compositions often have the drawback of impairing the cosmetic properties of the hair. Thus, the hair may become coarse or difficult to disentangle, and it may lose its pleasant feel and look or it may lack body. Moreover, the hair compositions may give rise to a powdering effect, giving the user a neglected appearance.

There is thus a need to find cosmetic compositions, especially styling compositions, which do not have the set of drawbacks mentioned above and which can give satisfactory fixing of the hairstyle, and can afford good cosmetic properties, such as a pleasant feel or a natural look.

The inventors have discovered, surprisingly and unexpectedly, that when grafted polysaccharide skeletons defined herein, are combined with fixing polymers defined herein, it is possible to obtain cosmetic compositions that satisfy at least one of the requirements mentioned above.

One subject of the invention is a cosmetic composition comprising, in a cosmetically acceptable medium, (a) at least one polymer comprising a polysaccharide skeleton grafted with at least one group comprising at least one polysiloxane, and (b) at least one non-silicone, non-cellulosic fixing polymer.

Another subject of the invention comprises a method of preparing a composition, comprising dissolving, in an organic solvent, (a) a polysaccharide containing carboxyl groups, and (b) a polysiloxane containing an epoxy end group and corresponding to formula (I), defined below, for a time and under conditions sufficient to obtain a polysaccharide and polysiloxane mixture. The method further comprises heating the mixture to a temperature of between 60 and 200° C. inclusive; reacting the carboxyl groups of the polysaccharide with the epoxy groups of the polysiloxane for a time and under conditions sufficient to form a polymer comprising a polysaccharide skeleton grafted with at least one group comprising at least one polysiloxane; and adding at least one non-silicone, non-cellulosic fixing polymer to the grafted polymer.

Another subject of the present invention relates to a cosmetic process, such as a hair process, for fixing and/or holding a hairstyle, comprising applying to hair, an effective amount of a composition comprising, in a cosmetically acceptable medium, (a) at least one polymer comprising a polysaccharide skeleton grafted with at least one group comprising at least one polysiloxane, and (b) at least one non-silicone, non-cellulosic fixing polymer.

Yet another subject of the invention relates to a method for holding and/or shaping a hairstyle, comprising applying to hair, an effective amount of a composition comprising, in a cosmetically acceptable medium (a) at least one polymer comprising a polysaccharide skeleton grafted with at least one group comprising at least one polysiloxane, and (b) at least one non-silicone, non-cellulosic fixing polymer.

The polymers comprising at least one polymer comprising a polysaccharide skeleton grafted with at least one group comprising at least one polysiloxane, in accordance with the present invention, comprise a principal chain formed from polysaccharide(s), onto which is grafted, and also optionally attached to at least one of its ends, at least one group comprising at least one polysiloxane.

These polymers comprising a polysaccharide skeleton grafted with at least one group comprising at least one polysiloxane, may be obtained according to any means known to those skilled in the art, such as by a reaction between: (i) a starting polysiloxane macromer functionalized on the polysiloxane chain; and (ii) at least one polysaccharide, which can be functionalized with a group that is capable of reacting with the functional group(s) borne by the said silicone to form a covalent bond.

Exemplary polysaccharide grafted polymers are described in U.S. Pat. No. 6,066,727, by the company Shin-Etsu, the disclosure of which is specifically incorporated by reference herein. These are copolymers obtained by reaction between a polysaccharide containing carboxyl groups and a polysiloxane containing an epoxy end group, in an organic solvent, optionally in the presence of a catalyst.

In one embodiment, the process for preparing these polymers comprises:

(a) dissolving, in an organic solvent, (a) a polysaccharide containing carboxyl groups, and (b) a polysiloxane containing an epoxy end group, and corresponding to formula (1) of a polysiloxane below:

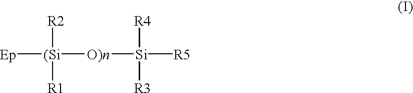

in which

n is an integer between 3 and 500 inclusive,

R₁, R₂, R₃, R₄ and R₅ are chosen, independently of each other, from monovalent C₁, to C₁₀ hydrocarbons and monovalent C₁, to C₁₀ halohydrocarbons, and

Ep is 2-(3,4-epoxycyclohexyl)ethyl; and

(b) heating this mixture of polysaccharides and polysiloxanes to a temperature between 60 and 200° C. inclusive, so as to make the carboxyl groups of the polysaccharides react with the epoxy groups of the polysiloxanes.

Exemplary polysaccharides suitable for carrying out step (a) of this process, include polysaccharides containing at least one group chosen from carboxylic, benzoyl, and succinoyl groups, such as hydroxypropylmethylcellulose phthalate and hydroxypropylmethylcellulose acetate succinate, and also include carboxymethylethylcellulose, and the polysaccharide pullulan acetate phthalate.

R₁, R₂, R₃, R₄ and R₅ can be chosen, independently of each other, from methyl, ethyl, propyl and butyl groups, C₃ to C₈ cycloalkyl groups such as cyclopentyl and cyclohexyl radicals, aryl groups such as phenyl and tolyl radicals, C₃ to C₈ aralkyl groups, such as benzyl and phenethyl radicals, and alkenyl groups, such as vinyl and allyl groups. These monovalent hydrocarbon radicals may optionally be totally or partially substituted, such as with a halogen atom, for example, with chloromethyl and 3,3,3-trifluoropropyl radicals.

Exemplary organic solvents suitable for performing step (a) of the process, include solvents chosen from ketones, for instance acetone, and cyclohexanone.

Step (b) can be carried out with stirring, optionally under an inert atmosphere.

The composition can comprise, as a relative percentage by weight of the composition, from 0.1% to 20% of the grafted polysaccharides, such as from 0.5% to 10% of the grafted polysaccharide.

The composition can comprise, as a relative percentage by weight of the composition, from 0.01% to 20% of fixing polymer, such as from 0.05% to 10% of fixing polymer.

The non-silicone and non-cellulosic fixing polymers that can be suitable in the invention include those generally used in the art. The fixing polymers can be chosen from cationic, anionic, amphoteric and nonionic polymers and mixtures thereof.

The non-silicone and non-cellulosic fixing polymers may be soluble in the cosmetically acceptable medium or insoluble in this same medium, and can be used in the form of dispersions of solid or liquid polymer particles (latex or pseudolatex).

The expression “non-cellulosic fixing polymer” means any fixing polymer free of cellobiose units.

The expression “non-silicone fixing polymer” means any fixing polymer free of silicon atoms in its structure.

The cationic fixing polymers which may be used according to the present invention can be chosen from polymers containing amine groups chosen from primary, secondary, tertiary and quaternary amine groups forming part of the polymer chain or directly attached thereto. The cationic fixing polymers can have a number-average molecular weight of between 500 and about 5 000 000, inclusive, such as between 1 000 and 3 000 000, inclusive.

Exemplary cationic polymers include:

(1) homopolymers and copolymers derived from monomers chosen from acrylic and methacrylic esters and amides, and containing at least one unit of the following formulae:

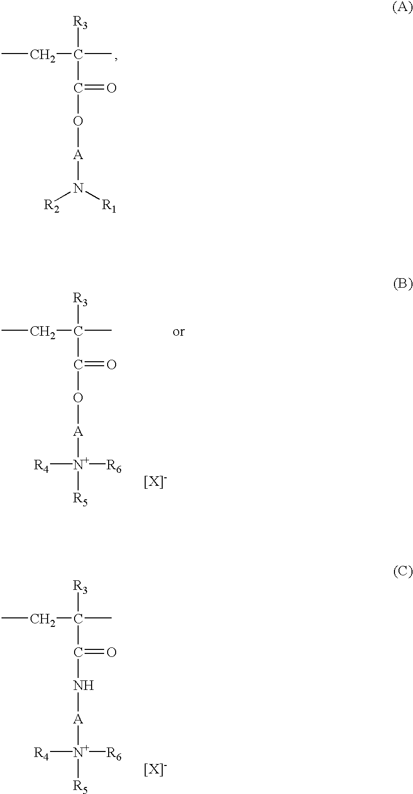

in which:

R₁ and R₂, which may be identical or different, are each chosen from hydrogen and alkyl groups having from 1 to 6 carbon atoms;

R₃ is chosen from hydrogen and CH₃;

A is chosen from linear and branched alkyl groups comprising from 1 to 6 carbon atoms, and hydroxyalkyl groups comprising from 1 to 4 carbon atoms;

R₄, R₅ and R₆, which may be identical or different, are each chosen from alkyl groups having from 1 to 18 carbon atoms, and benzyl groups;

[X]⁻ is chosen from methosulphate anions and halides, such as chloride and bromide.

The copolymers of the family (1) can additionally comprise one or more units derived from comonomers which can be chosen from acrylamides, methacrylamides, diacetone acrylamides, acrylamides and methacrylamides substituted on the nitrogen with C₁₋₄ alkyl groups, groups derived from acrylic and methacrylic acids and esters thereof, vinyllactams such as vinylpyrrolidone and vinylcaprolactam, and vinyl esters.

Thus, among these copolymers of the family (1), mention may be made of:

copolymers derived from acrylamide and dimethylaminoethyl methacrylate quaternized with groups chosen from dimethyl sulphate and dimethyl halides,

copolymers derived from acrylamide and methacryloyloxyethyltrimethyl-ammonium chloride, described, for example, in European patent application EP-A-080 976, the disclosure of which is specifically incorporated by reference herein.

copolymers derived from acrylamides and methacryloyloxyethyltrimethylammonium methosulphate,

copolymers derived from quaternized and non-quaternized vinylpyrrolidone/dialkylaminoalkyl acrylates and methacrylates, such as products sold under the name “Gafquat®” by the company ISP, for example, “Gafquat® 734” or “Gafquat® 755”, and products known as “Copolymer® 845, 958 and 937”. These polymers are described in detail in French patents 2 077 143 and 2 393 573, the disclosures of which are specifically incorporated by reference herein.

dimethylaminoethyl methacrylate/vinylcaprolactam/vinylpyrrolidone terpolymers, such as the product sold under the name Gaffix® VC 713 by the company ISP, and

quaternized vinylpyrrolidone/dimethylaminopropyl methacrylamide copolymers, such as the product sold under the name “Gafquat® HS 100” by the company ISP.

(2) quaternized guar gums;

(3) quaternary copolymers derived from vinylpyrrolidone and vinylimidazole;

(4) chitosans and salts thereof; representative salts which may be used include chitosan acetate, lactate, glutamate, gluconate and pyrrolidone carboxylate.

Among these compounds, mention may be made of chitosan having a degree of deacetylation of 90% by weight, and the chitosan pyrrolidone carboxylate sold under the name Kytamer® PC by the company Amerchol.

The anionic fixing polymers generally used can include polymers comprising groups derived from monomers chosen from carboxylic acid, sulphonic acid and phosphoric acid and can have, for example, a number-average molecular mass of between 500 and 5 000 000 inclusive.

The carboxylic groups can be provided by monomers chosen from unsaturated monocarboxylic and dicarboxylic acid monomers, such as those corresponding to the formula:

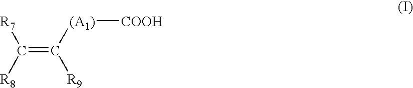

in which:

n is an integer from 0 to 10,

A₁ is chosen from methylene groups wherein at least one of said methylene groups can be bonded to a hetero atom, such as oxygen and sulphur,

R₇ is chosen from hydrogen, phenyl groups and benzyl groups,

R₈ is chosen from hydrogen, lower alkyls, and carboxyl groups,

R₉ is chosen from hydrogen, lower alkyls, —CH₂—COOH, phenyl groups, and benzyl groups.

In the above mentioned formula, a lower alkyl group denotes C₁-C₈ alkyls, such as alkyl groups having 1 to 4 carbon atoms, for example, methyl and ethyl.

Exemplary anionic fixing polymers comprising carboxylic groups include:

A) homo- and copolymers derived from acrylic and methacrylic acid monomers, and salts thereof, such as products sold under the names Versicol® E or K by the company Allied Colloid, and Ultrahold® by the company BASF; and copolymers derived from monomers chosen from acrylic acids, acrylamides, and sodium salts of polyhydroxycarboxylic acids.

B) copolymers derived from (a) monomers chosen from acrylic and methacrylic acid, and (b) monoethylenic monomers such as ethylene, styrene, vinyl esters, acrylic and methacrylic acid esters. These copolymers can be optionally grafted onto a polyalkylene glycol such as polyethylene glycol, and the graft polymers can be optionally crosslinked. Exemplary polymers are described in French Patent No. 1 222 944 and German patent application No. 2 330 956, the disclosures of which are specifically incorporated by reference herein. Copolymers containing an optionally N-alkylated and/or hydroxyalkylated acrylamide unit in their chain also fall within this class of polymers, and are described in Luxembourg patent application Nos. 75370 and 75371, the disclosures of which are specifically incorporated by reference herein. Mention may also be made of copolymers derived from acrylic acid and C₁-C₄ alkyl methacrylates, and terpolymers derived from vinylpyrrolidone, acrylic acid and C₁-C₂₀ alkyl methacrylates, for example lauryl, such as the product sold by the company ISP under the name Acrylidone® LM and methacrylic acid/ethyl acrylate/tert-butyl acrylate terpolymers such as the product sold under the name Luvimer® 100 P by the company BASF.

C) copolymers derived from crotonic acid, such as those containing vinyl acetate and propionate units, and optionally other monomers such as allylic esters, methallylic esters, and vinyl ethers and vinyl esters of linear and branched saturated carboxylic acids with a long hydrocarbon-based chain such as those containing at least 5 carbon atoms. These polymers can be grafted and crosslinked. The copolymers can also alternatively be derived from another monomer chosen from vinyl, allylic and methallylic esters of α- and β-cyclic carboxylic acids. Such polymers are described, inter alia, in French patent Nos. 1 222 944, 1 580 545, 2 265 782, 2 265 781, 1 564 110, and 2 439 798, the disclosures of which are specifically incorporated by reference herein. A commercial product falling into this class is the resin 28-29-30 sold by the company National Starch.

D) copolymers derived from C₄-C₈ monounsaturated carboxylic acids and anhydrides, the copolymers being chosen from:

copolymers derived from (i) at least one monomer chosen from maleic, fumaric, and itaconic acids and anhydrides, and (ii) at least one monomer chosen from vinyl esters, vinyl ethers, vinyl halides, phenylvinyl derivatives, acrylic acids and its esters. The anhydride functions of these copolymers can be monoesterified or monoamidated. Exemplary polymers are described in U.S. Pat. Nos. 2,047,398, 2,723,248, and 2,102,113, and GB patent No. 839 805, the disclosures of which are specifically incorporated by reference herein. Exemplary commercial products include those sold under the names Gantrez® AN or ES by the company ISP,

copolymers derived from (i) at least one monomer chosen from maleic, citraconic, and itaconic anhydrides, and (ii) at least one monomer chosen from allylic and methallylic esters optionally containing at least one group chosen from acrylamides, methacrylamides, α-olefin groups, acrylic esters, methacrylic esters, acrylic acids, methacrylic acids, and vinylpyrrolidone. The anhydride functions of these copolymers can be monoesterified or monoamidated. Exemplary polymers are described in French patent Nos. 2 350 384 and 2 357 241, the disclosures of which are specifically incorporated by reference herein.

E) polyacrylamides containing carboxylate groups; and

F) anionic polyurethanes, such as the product sold by BASF under the name Luviset PUR.

The polymers comprising sulphonic groups include, for example, polymers comprising units chosen from vinylsulphonic, styrenesulphonic, naphthalenesulphonic or acrylamidoalkylsulphonic units. These polymers can be chosen from:

polyvinylsulphonic acid salts having a number average molecular weight ranging from 1 000 to 100 000, and copolymers derived from an unsaturated comonomer, such as acrylic and methacrylic acids and their esters, acrylamides and their derivatives, vinyl ethers, and vinylpyrrolidone;

polystyrenesulphonic acid salts, such as the sodium salts sold for example under the name Flexan® 130 by National Starch. Exemplary compounds are described in patent FR 2 198 719, the disclosure of which is specifically incorporated by reference herein;

polyacrylamidesulphonic acid salts, such as polyacrylamidoethylpropanesulphonic acid and those mentioned in U.S. Pat. No. 4,128,631, the disclosure of which is specifically incorporated by reference herein.

In one embodiment, the anionic fixing polymers are chosen from acrylic acid copolymers, such as: acrylic acid/ethyl acrylate/N-tert-butylacrylamide terpolymers sold under the name Ultrahold® Strong by the company BASF; copolymers derived from crotonic acid, such as vinyl acetate/vinyl tert-butylbenzoate/crotonic acid terpolymers and the crotonic acid/vinyl acetate/vinyl neododecanoate terpolymers sold under the name Resin 28-29-30 by the company National Starch; polymers derived from at least one monomer chosen from maleic, fumaric, itaconic acids and anhydrides and at least one monomer chosen from vinyl esters, vinyl ethers, vinyl halides, phenylvinyl derivatives and acrylic acid and esters thereof, such as the methyl vinyl ether/monoesterified maleic anhydride copolymers sold, for example, under the name Gantrez® by the company ISP; copolymers derived from methacrylic acid and methyl methacrylate, sold under the name Eudragit® L by the company Rohm Pharma; copolymers derived from methacrylic acid and ethyl acrylate, such as those sold under the name Luvimer® MAEX or MAE by the company BASF; vinyl acetate/crotonic acid copolymers and the vinyl acetate/crotonic acid copolymers grafted with polyethylene glycol, such as those sold under the name Aristoflex® A by the company BASF; and the polyurethane Luviset PUR®, sold by the company BASF.

Exemplary anionic fixing polymers include those chosen from methyl vinyl ether/monoesterified maleic anhydride copolymers, such as those sold under the name Gantrez® ES 425 by the company ISP; acrylic acid/ethyl acrylate/N-tert-butylacrylamide terpolymers, such as those sold under the name Ultrahold® Strong by the company BASF; copolymers derived from methacrylic acid and methyl methacrylate, such as those sold under the name Eudragit® L by the company Rohm Pharma; vinyl acetate/vinyl tert-butylbenzoate/crotonic acid terpolymers and crotonic acid/vinyl acetate/vinyl neododecanoate terpolymers, such as those sold under the name Resin 28-29-30 by the company National Starch; copolymers derived from methacrylic acid and ethyl acrylate, such as those sold under the name Luvimer® MAEX and MAE by the company BASF; vinylpyrrolidone/acrylic acid/lauryl methacrylate terpolymers, such as those sold under the name Acrylidone® LM by the company ISP; and the polyurethane Luviset PUR® sold by the company BASF.

In one embodiment, the amphoteric fixing polymers can be chosen from polymers comprising units B and C distributed randomly in the polymer chain, in which B is derived from a monomer containing at least one basic nitrogen atom, and C is derived from an acid monomer containing at least one group chosen from carboxylic and sulphonic groups. Alternatively B and C can be derived from zwitterionic monomers chosen from carboxybetaines and sulphobetaines.

B and C can also comprise a cationic polymer chain containing groups chosen from primary, secondary, tertiary and quaternary amine groups, in which at least one of the amine groups bears a group chosen from carboxylic and sulphonic groups connected via a hydrocarbon-based group. Alternatively, B and C can form part of a chain of a polymer containing an a-dicarboxylic ethylene unit in which one of the carboxylic groups has been made to react with a polyamine containing at least one group chosen from primary and secondary amine groups.

Exemplary amphoteric fixing polymers in these embodiments include those chosen from:

(1) polymers resulting from the copolymerization of (i) monomers derived from a vinyl compound bearing a carboxylic group, such as monomers chosen from acrylic acids, methacrylic acids, maleic acids, a-chloroacrylic acids, and (ii) basic monomers derived from a substituted vinyl compound containing at least one basic atom, such as those monomers chosen from dialkylaminoalkyl methacrylates and acrylates, and dialkylaminoalkylmethacrylamides and -acrylamides. Exemplary polymers are described in U.S. Pat. No. 3,836,537, the disclosure of which is specifically incorporated by reference herein. Mention may also be made of the sodium acrylate/acrylamidopropyltrimethylammonium chloride copolymer sold under the name Polyquart KE 3033 by the company Henkel.

The vinyl compound may also be a dialkyldiallylammonium salt such as diethyidiallylammonium chloride. Exemplary copolymers derived from acrylic acid and the latter monomer are sold under the names Merquat 280, Merquat 295 and Merquat Plus 3330 by the company Calgon.

(2) polymers derived from:

a) at least one monomer chosen from acrylamides and methacrylamides substituted on the nitrogen atom with an alkyl group,

b) at least one acidic comonomer containing at least one reactive carboxylic group, and

c) at least one basic comonomer, such as esters containing primary, secondary, tertiary and quaternary amine substituents of acrylic and methacrylic acids, and the product of quaternization of dimethylaminoethyl methacrylate with dimethyl or diethyl sulphate.

Exemplary N-substituted acrylamides and methacrylamides include those compounds in which the alkyl groups contain from 2 to 12 carbon atoms, such as N-ethylacrylamide, N-tert-butylacrylamide, N-tert-octylacrylamide, N-octylacrylamide, N-decylacrylamide, N-dodecylacrylamide and the corresponding methacrylamides.

Exemplary acidic comonomers include those chosen from acrylic acid, methacrylic acid, crotonic acid, itaconic acid, maleic acid and fumaric acid and alkyl monoesters, having 1 to 4 carbon atoms, of maleic and fumaric acids and anhydrides.

Exemplary basic comonomers include those chosen from aminoethyl, butylaminoethyl, N,N′-dimethylaminoethyl and N-tert-butylaminoethyl methacrylates.

The copolymers whose CTFA (4th edition, 1991) name is octylacrylamide/acrylates/butylaminoethyl methacrylate copolymer, such as the products sold under the name Amphomer® and Lovocryl® 47 by the company National Starch, can be used.

(3) crosslinked and acylated polyamino amides partially or totally derived from polyamino amides of general formula:

—[CO—R₁₀—CO—Z]— (II)

in which R₁₀ is chosen from divalent groups derived from saturated dicarboxylic acids, mono- and dicarboxylic aliphatic acids containing an ethylenic double bond, esters of these acids of a lower alkanol having 1 to 6 carbon atoms inclusive, and a group derived from the addition of any one of the said acids to an amine chosen from bis(primary) and bis(secondary) amines, and

Z is chosen from a group derived from bis(primary), mono- and bis(secondary) polyalkylene-polyamines. Z can be chosen from:

a) in proportions of from 60 to 100 mol %, the group

—NH—[(CH₂)_{X}—NH]ₚ— (IV)

where x=2 and p=2 or 3, or alternatively x=3 and p=2, this group being derived from amines chosen from diethylenetriamines, triethylenetetraamines, and dipropylenetriamines;

b) in proportions of from 0 to 40 mol %, the group (IV) above in which x=2 and p=1 and which is derived from ethylenediamine, or the group derived from piperazine:

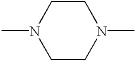

c) in proportions of from 0 to 20 mol %, the —NH—(CH₂)₆—NH— group derived from hexamethylenediamine. These polyamino amines can be crosslinked by reaction addition of a difunctional crosslinking agent chosen from epihalohydrines, diepoxides, dianhydrides and bis-unsaturated derivatives, by using from 0.025 to 0.35 mol of crosslinking agent per amine group of the polyamino amide and acylated by the action of acrylic acids, chloroacetic acids alkane sultones, and salts thereof.

Exemplary saturated carboxylic acids are chosen from acids having 6 to 10 carbon atoms, such as adipic acid, 2,2,4trimethyladipic acid and 2,4,4-trimethyladipic acid. Other representative carboxylic acids include terephthalic acid, acids containing an ethylenic double bond such as, for example, acrylic acid, methacrylic acid and itaconic acid.

The alkane sultones used in the acylation can be chosen from propane sultone and butane sultone. The salts of the acylating agents can be chosen from sodium and potassium salts.

(4) polymers containing zwitterionic units derived from at least one monomer of formula:

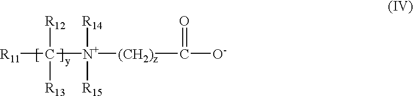

in which:

R₁₁ is chosen from polymerizable unsaturated groups, such as acrylates, methacrylates, acrylamides and methacrylamides,

y and z are chosen from an integer from 1 to 3,

R₁₂ and R₁₃ are chosen from hydrogen, methyl, ethyl and propyl groups,

R₁₄ and R₁₅ are chosen from hydrogen and alkyl groups such that the sum of the carbon atoms in R₁₄ and R₁₅ does not exceed 10.

The polymers comprising such units can also contain units derived from non-zwitterionic monomers such as those monomers chosen from dimethyl and diethylaminoethyl acrylates and methacrylates, alkyl acrylates and methacrylates, acrylamides and methacrylamides, and vinyl acetate.

By way of example, mention may be made of butyl methacrylate/N,N-dimethylcarboxyaminoethyl methacrylate copolymers.

(5) polymers comprising chitosan containing monomer units corresponding to the following formulae:

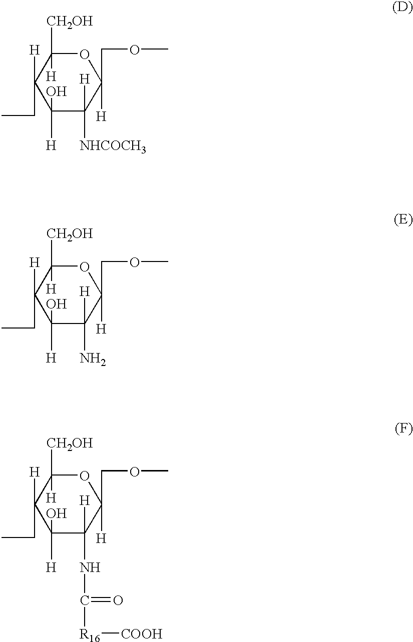

the unit (D) being present in proportions of between 0 and 30% inclusive, the unit (E) in proportions of between 5 and 50% inclusive and the unit (F) in proportions of between 30 and 90% inclusive, it being understood that, in this unit (F), R₁₆ is a group of formula:

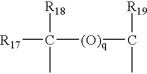

in which:

if q=0, then R₁₇, R₁₈ and R₁₉, which may be identical or different, are chosen from hydrogen, methyl, hydroxyl, acetoxy, amino, monoalkylamines and dialkylamines, the alkyl portion of which may be optionally interrupted by at least one nitrogen atom, and/or optionally substituted with at least one amine, hydroxyl, carboxyl, alkylthio and sulphonic groups, and an alkylthio group in which the alkyl group bears an amino group; at least one of the groups R₁₇, R₁₈ and R₁₉ being, in this case, hydrogen;

or, if q=1, R₁₇, R₁₈ and R₁₉, which may be identical or different, are chosen from hydrogen, and

acid and base addition salts of said polymers comprising said chitosan containing monomer units.

(6) polymers corresponding to the general formula (V), examples of which are described in French patent 1 400 366, the disclosure of which is specifically incorporated by reference herein:

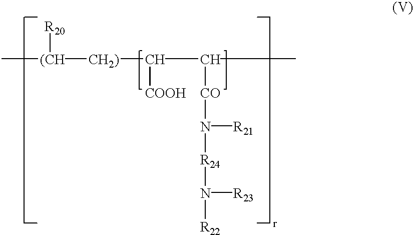

in which:

R₂₀ is chosen from hydrogen, CH₃O, CH₃CH₂O and phenyl,

R₂₁ is chosen from hydrogen and C₁-C₆ alkyl groups, such as methyl and ethyl,

R₂₂ is chosen from hydrogen and C₁-C₆ alkyl groups, such as methyl and ethyl,

R₂₃ is chosen from C₁-C₆ alkyl groups, such as methyl and ethyl, and groups corresponding to the formula: —R₂₄—N(R₂₂)₂, wherein R₂₄ is chosen from —CH₂—CH₂—, —CH₂—CH₂—CH₂—, and —CH₂—CH(CH₃)—, and R₂₂ is defined as above, and

r is chosen such that the number-average molecular weight of said polymer ranges from 500 to 6,000,000, such as from 1000 to 1,000,000.

(7) polymers derived from the N-carboxyalkylation of chitosan, such as N-carboxymethylchitosan and N-carboxybutylchitosan.

(8) amphoteric polymers of the type —D—X—D—X chosen from:

a) polymers obtained by the action of a reactant, chosen from chloroacetic acid and sodium chloroacetate, on compounds comprising at least one unit of formula:

—D—X—D—X—D— (VI)

wherein D is

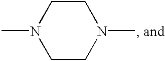

X is chosen from the symbols E and E′, wherein E and E′, which may be identical or different, are each chosen from divalent alkylene groups comprising at least one chain chosen from linear and branched chains comprising up to 7 carbon atoms in the principal chain, wherein said divalent alkylene groups are optionally substituted with at least one hydroxyl group. E and E′ can additionally comprise: a hetero atom chosen from oxygen, nitrogen, sulphur; and 1 to 3 rings chosen from aromatic and heterocyclic rings. The oxygen, nitrogen and sulphur atoms can be present in the form of at least one group chosen from ether, thioether, sulphoxide, sulphone, sulphonium, alkylamine and alkenylamine groups, hydroxyl, benzylamine, amine oxide, quaternary ammonium, amide, imide, alcohol, ester and urethane groups.

b) polymers of formula:

—D—X—D—X— (VI′)

wherein D is

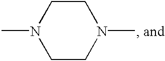

X is chosen from the symbols E and E′ and wherein at least one X is chosen from E′;

E is chosen from divalent alkylene groups comprising at least one chain chosen from linear and branched chains comprising up to 7 carbon atoms in the principal chain, wherein said divalent alkylene groups are optionally substituted with at least one hydroxyl group. E can additionally comprise: a hetero atom chosen from oxygen, nitrogen, sulphur; and 1 to 3 rings chosen from aromatic and heterocyclic rings. The oxygen, nitrogen and sulphur atoms can be present in the form of at least one group chosen from ether, thioether, sulphoxide, sulphone, sulphonium, alkylamine and alkenylamine groups, hydroxyl, benzylamine, amine oxide, quaternary ammonium, amide, imide, alcohol, ester and urethane groups, and

E′ is chosen from divalent alkylene groups comprising at least one chain chosen from linear and branched chains comprising up to 7 carbon atoms in the principal chain, wherein said divalent alkylene groups are optionally substituted with at least one hydroxyl group. E′ can comprise at least one nitrogen atom substituted with an alkyl chain which can be interrupted by an oxygen atom and, wherein said alkyl chain comprises at least one functional group chosen from at least one carboxyl function and hydroxyl functional groups, and wherein said at least one alkyl chain is betainized by reaction with a reactant chosen from chloroacetic acid and sodium chloroacetate.

(9) (C₁-C₅)alkyl vinyl ether/maleic anhydride copolymers partially modified by reactions chosen from semiamidation with an N,N-dialkylaminoalkylamine such as N,N-dimethylaminopropylamine, and from semiesterification with an N,N-dialkylaminoalkynol. These copolymers can also contain other vinyl comonomers such as vinylcaprolactam.

Representative amphoteric fixing polymers include those of the family (3), such as the copolymers whose CTFA name is octylacrylamide/acrylates/butylaminoethyl methacrylate copolymer, such as the products sold under the names Amphomer®, Amphomer® LV 71 and Lovocryl® 47 by the company National Starch, and those of the family (4) such as butyl methacrylate/N,N-dimethylcarboxyaminoethyl methacrylate copolymers.

The nonionic fixing polymers can be chosen, for example, from:

polyalkyloxazolines;

vinyl acetate homopolymers;

copolymers derived from vinyl acetate and acrylicester;

copolymers derived from vinyl acetate and ethylene;

copolymers derived from vinyl acetate and maleic ester, for example, dibutyl maleate;

copolymers derived from acrylic esters, such as, for example, copolymers derived from alkyl acrylates and alkyl methacrylates, such as the products sold by the company Rohm & Haas under the names Primal® AC-261 K and Eudragit® NE 30 D, by the company BASF under the name 8845, or by the company Hoechst under the name Appretan® N9212;

copolymers derived from acrylonitrile and a nonionic monomer chosen, for example, from butadiene, alkyl acrylates and alkyl methacrylates; mention may be made of the products sold under the name CJ 0601 B by the company Rohm & Haas;

styrene homopolymers;

copolymers derived from styrene and at least one monomer chosen from alkyl acrylates and alkyl methacrylates, such as the products Mowilith® LDM 6911, Mowilith® DM 611 and Mowilith® LDM 6070 sold by the company Hoechst, and the products Rhodopas® SD 215 and Rhodopas® DS 910 sold by the company Rhône-Poulenc;

copolymers derived from styrene, alkyl methacrylates and alkyl acrylates;

nonionic polyurethanes;

copolymers derived from styrene and butadiene;

copolymers derived from styrene, butadiene and vinylpyridine;

copolymers derived from alkyl acrylates and urethanes;

polyamides; and

vinyl lactam homopolymers and copolymers.

The alkyl groups in the nonionic polymers mentioned above can comprise from 1 to 6 carbon atoms.

In one embodiment, the fixing polymers are nonionic polymers. In another embodiment, the fixing polymers are nonionic polymers derived from vinyl lactam units. Such exemplary nonionic polymers are described in U.S. Pat. Nos. 3,770,683, 3,929,735, 4,521,504, 5,158,762 and 5,506,315 and in patent publication nos. WO 94/121148, WO 96/06592 and WO 96/10593, the disclosures of which are specifically incorporated by reference herein.

The fixing polymers may be in pulverulent form or in the form of a solution or suspension.

In one embodiment, the homopolymers or copolymers containing vinyl lactam units comprise units of formula:

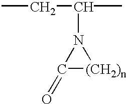

in which n is independently 3, 4 or 5.

The number-average molecular weight of the polymers containing vinyl lactam units can be greater than about 5 000, such as between 10 000 and 1 000 000, inclusive, and also such as between 10 000 and 100 000 inclusive.

Fixing polymers that may be used include polyvinylpyrrolidones such as those sold under the name Luviskol® K30 by the company BASF; polyvinylcaprolactams such as those sold under the name Luviskol® PLUS by the company BASF; poly(vinylpyrrolidone/vinyl acetate) copolymers such as those sold under the name PVPVA® S630L by the company ISP, Luviskol® VA 73, VA 64, VA 55, VA 37 and VA 28 by the company BASF; and poly(vinylpyrrolidone/vinyl acetate/vinyl propionate) terpolymers such as, for example, those sold under the name Luviskol® VAP 343 by the company BASF.

The cosmetically acceptable medium can comprise at least one solvent chosen from water and cosmetically acceptable solvents, such as alcohols and water-solvent mixtures. Exemplary solvents include C₁-C₄ alcohols, aliphatic and aromatic ketones, esters of C₁-C₈ short chain and C₉-C₂₀ long chain acids and of C₁-C₈ and C₉-C₂₀ alcohols, pentane, heptane, polyols, polyol ethers and isodecane.

Other exemplary solvents include at least one alcohol chosen from ethanol and isopropanol.

The ratio between the relative concentration by weight of the polymers containing polysaccharide skeletons, and the relative concentration by weight of the fixing polymers, can range from 0.01:1 to 100:1, such as ratios ranging from 0.05:1 to 20:1 and also from 0.1:1 to 10:1.

The composition of the invention may also contain at least one additive chosen from: anionic, cationic, nonionic and amphoteric surfactants; fragrances; screening agents; preserving agents; proteins; vitamins; provitamins; polymers other than those of the invention; plant, mineral and synthetic oils; polyols such as glycols and glycerol; silicones; fatty alcohols; and any other additive conventionally used in cosmetic compositions.

A person skilled in the art can take care to select the optional compound(s) to be added to the composition according to the invention such that the advantageous properties intrinsically associated with the composition in accordance with the invention are not adversely affected by the envisaged addition.

The compositions in accordance with the invention may be applied to the skin, the nails, the lips, the hair, the eyebrows and the eyelashes.

The compositions in accordance with the invention may be suitable for wet or dry hair, as styling products.

The invention will be illustrated more fully with the aid of the non-limiting example, which follows.

All the percentages are relative percentages by weight relative to the total weight of the composition, and a.m. means active material.

### EXAMPLE

A composition in accordance with the invention is prepared from the ingredients listed below.

Composition 1:

| | |
|---|---|
| Cellulose containing silicone grafts [1] | 3% |
| Ultrahold Strong [2] | 3% |
| Water | 10% |
| Ethanol qs for | 100% |

[1] Hydroxypropylmethylcellulose acetate succinate containing polydimethylsiloxane grafts, as synthesized in Example No. 2 of U.S. Pat. No. US 6,066,727 by the company Shin Etsu, the disclosure of which is specifically incorporated by reference herein.
[2] Acrylates/butylacrylamide copolymer sold by the company BASF.

[1] Hydroxypropylmethylcellulose acetate succinate containing polydimethylsiloxane grafts, as synthesized in Example No. 2 of U.S. Pat. No. 6,066,727 by the company Shin Etsu, the disclosure of which is specifically incorporated by reference herein.

[2] Acrylates/butylacrylamide copolymer sold by the company BASF.

The composition is applied to locks of European chestnut-brown hair. It gives very good results in terms of lacquering power and cosmetic effects, and in terms of disentangling, softness and feel, while at the same time giving rise to little powdering.

What is claimed is:

1. A cosmetic composition comprising, in a cosmetically acceptable medium:
(a) at least one polymer comprising a polysaccharide skeleton grafted with at least one group comprising at least one polysiloxane; and
(b) at least one non-silicone, non-cellulosic fixing polymer.

2. The composition according to claim 1, wherein at least one group comprising at least one polysiloxane is attached to at least one end of the polysaccharide skeleton.

3. The composition according to claim 1, wherein the at least one fixing polymer is chosen from cationic, anionic, amphoteric and nonionic polymers, and mixtures thereof.

4. The composition according to claim 3, wherein the at least one fixing polymer is an anionic fixing polymer containing carboxylic groups, the at least one fixing polymer being chosen from:
A) homo- and copolymers derived from acrylic and methacrylic acid monomers and salts thereof, copolymers derived from acrylic acid and acrylamide, and sodium salts of polyhydroxycarboxylic acids;
B) copolymers derived from (a) monomers chosen from acrylic and methacrylic acid, and (b) monoethylenic monomers chosen from ethylene, styrene, vinyl esters, and acrylic and methacrylic acid esters; the copolymers being optionally grafted onto a polyalkylene glycol; and the graft copolymers being optionally crosslinked;
C) copolymers derived from crotonic acid;
D) copolymers derived from C₄-C₈ monounsaturated carboxylic acids and anhydrides, the copolymers being chosen from:
copolymers derived from (i) at least one monomer chosen from maleic, fumaric and itaconic acids and anhydrides, and (ii) at least one monomer chosen from vinyl esters, vinyl ethers, vinyl halides, phenylvinyl derivatives, acrylic acid and its esters, the anhydride functions of these copolymers optionally being monoesterified or monoamidated; and
copolymers derived from (i) at least one monomer chosen from maleic, citraconic and itaconic anhydrides, and (ii) at least one monomer chosen from allylic and methallylic esters optionally containing at least one group chosen from acrylamides, methacrylamides, α-olefins, acrylic and methacrylic esters, acrylic and methacrylic acids and vinylpyrrolidones;
E) polyacrylamides containing carboxylate groups; and
F) anionic polyurethanes.

5. The composition according to claim 4, wherein the copolymers B) are derived from monomers optionally grafted onto polyethylene glycol.

6. The composition according to claim 3, wherein the at least one fixing polymer is a nonionic polymer chosen from:
polyalkyloxazolines;
vinyl acetate homopolymers;
copolymers derived from vinyl acetate and acrylic ester;
copolymers derived from vinyl acetate and ethylene;
copolymers derived from vinyl acetate and maleic ester
copolymers derived from acrylic esters;
copolymers derived from acrylonitrile and a nonionic monomer;
styrene homopolymers;
copolymers derived from styrene and at least one monomer chosen from alkyl acrylates and alkyl methacrylates;
copolymers derived from styrene, alkyl methacrylate and alkyl acrylate;
nonionic polyurethanes;
copolymers derived from styrene and butadiene;
copolymers derived from styrene, butadiene and vinylpyridine;
copolymers derived from alkyl acrylate and urethane;
polyamides; and
vinyl lactam homopolymers and copolymers.

7. The composition according to claim 6, wherein the at least one fixing polymer is a nonionic polymer chosen from copolymers derived from vinyl acetate and dibutyl maleate.

8. The composition according to claim 1, wherein a ratio of the relative concentration by weight of the at least one polymer to the relative concentration by weight of the at least one fixing polymer ranges from 0.01:1 to 100:1.

9. The composition according to claim 8, wherein the ratio of the relative concentration by weight of the at least one polymer to the relative concentration by weight of the at least one fixing polymer ranges from 0.05:1 to 20:1.

10. The composition according to claim 9, wherein the ratio of the relative concentration by weight of the at least one polymer to the relative concentration by weight of the at least one fixing polymer ranges from 0.1:1 to 10:1.

11. The composition according to claim 1, wherein the composition comprises, as a relative percentage by weight of the composition, from 0.1% to 20% of the at least one polymer.

12. The composition according to claim 11, wherein the composition comprises, as a relative percentage by weight of the composition, from 0.5% to 10% of the at least one polymer.

13. The composition according to claim 1, wherein the composition comprises, as a relative percentage by weight of the composition, from 0.01% to 20% of the at least one fixing polymer.

14. The composition according to claim 13, wherein the composition comprises, as a relative percentage by weight of the composition, from 0.05% to 10% of the at least one fixing polymer.

15. The composition according to claim 1, wherein the composition further comprises at least one additive chosen from: anionic, cationic, nonionic and amphoteric surfactants; fragrances; screening agents; preserving agents; proteins; vitamins; provitamins; polymers other than said at least one polymer and said at least one fixing polymer; plant, mineral and synthetic oils; polyols; silicones; fatty alcohols and any other additive conventionally used in cosmetic compositions.

16. The composition according to claim 15, wherein the polyols comprise at least one polyol chosen from glycols and glycerol.

17. A method of preparing a composition, comprising:
dissolving, in an organic solvent, (a) a polysaccharide containing carboxyl groups, and (b) a polysiloxane containing an epoxy end group and corresponding to formula (I), for a time and under conditions sufficient to obtain a polysaccharide and polysiloxane mixture: 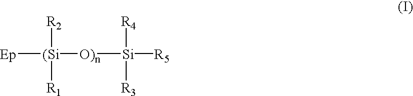
in which:
n is an integer between 3 and 500 inclusive,
R₁, R₂, R₃, R₄ and R₅ are chosen, independently of each other, from monovalent C₁ to C₁₀ hydrocarbons and monovalent C₁ to C₁₀ halohydrocarbons, and
Ep is 2-(3,4-epoxycyclohexyl)ethyl;
heating the mixture to a temperature of between 60 and 200° C. inclusive;
reacting the carboxyl groups of the polysaccharide with the epoxy groups of the polysiloxane for a time and under conditions sufficient to form a polymer comprising a polysaccharide skeleton grafted with at least one group comprising at least one polysiloxane; and
adding at least one non-silicone, non-cellulosic fixing polymer to the grafted polymer to prepare said composition.

18. The method according to claim 17, wherein the polysaccharide (a) is chosen from polysaccharides comprising carboxylic, benzoyl and succinoyl groups.

19. The method according to claim 18, wherein the polysaccharide (a) is chosen from hydroxypropylmethylcellulose phthalate, hydroxypropylmethylcellulose acetate succinate, carboxymethylethylcellulose, and pullulan acetate phthalate.

20. The method according to claim 17, wherein R₁, R₂, R₃, R₄ and R₅ are chosen, independently of each other, from methyl, ethyl, propyl, butyl, C₃-C₈ cycloalkyls, aryl, C₃-C₈ aralkyls, and alkenyl groups.

21. A cosmetic hair process for fixing and/or holding a hairstyle, comprising:
applying to hair, an effective amount of a composition comprising, in a cosmetically acceptable medium:
(a) at least one polymer comprising a polysaccharide skeleton grafted with at least one group comprising at least one polysiloxane; and
(b) at least one non-silicone, non-cellulosic fixing polymer.

22. A method for holding and/or shaping a hairstyle, comprising:
applying to the hair, an effective amount of a composition comprising, in a cosmetically acceptable medium:
(a) at least one polymer comprising a polysaccharide skeleton A grafted with at least one group comprising at least one polysiloxane; and
(b) at least one non-silicone, non-cellulosic fixing polymer.
